(19) United States
(12) Patent Application Publication
(10) Pub. No.: US 2026/0083955 A1
(43) Pub. Date: Mar. 26, 2026

(54) PERCUTANEOUS BLOOD PUMP OUTFLOW WINDOW MARKINGS

(71) Applicant: BOSTON SCIENTIFIC SCIMED, INC., Maple Grove, MN (US)

(72) Inventors: Judith Tiferes Wang, Shoreview, MN (US); Michael Kaland, White Bear Lake, MN (US); Corey N. Meyer, Zimmerman, MN (US); Christopher John Sperry, Plymouth, MN (US)

(73) Assignee: BOSTON SCIENTIFIC SCIMED, INC., Maple Grove, MN (US)

(21) Appl. No.: 19/333,680

(22) Filed: Sep. 19, 2025

Related U.S. Application Data
(60) Provisional application No. 63/697,149, filed on Sep. 20, 2024.

(51) Int. Cl. A61M 60/13; A61M 60/216; A61M 60/416; A61M 60/81

(52) U.S. Cl. CPC A61M 60/13; A61M 60/216; A61M 60/416; A61M 60/81; A61M 2205/583

(57) ABSTRACT

Various aspects of this disclosure are directed toward a percutaneous circulatory support device for use with a guidewire, the device comprising a housing having an interior lumen, an exterior surface, a blood inlet, and a blood outlet that includes at least one blood outflow window. An impeller is disposed within the interior lumen, with the impeller configured to rotate relative to the housing to cause blood flow into the blood inlet, through the interior lumen of the housing, and out of the blood outlet. A motor is operatively coupled to the impeller, the motor configured to rotatably drive the impeller, wherein the exterior surface of the housing adjacent the at least one blood outflow window comprises a marking.

### CROSS REFERENCE TO RELATED APPLICATIONS

The present application claims the benefit of U.S. Provisional Patent Application Ser. No. 63/697,149, filed Sep. 20, 2024, the disclosure of which is incorporated herein by reference.

### TECHNICAL FIELD

The present disclosure pertains to a percutaneous blood pump. More particularly, the present disclosure pertains to a percutaneous blood pump including a blood outlet with multiple windows for blood outflow, and markings on an outer surface of the blood outlet denoting which window(s) a user can load a guidewire through.

### BACKGROUND

A wide variety of medical devices have been developed for medical use including, for example, devices for delivering intravascular medical devices into a blood vessel of a patient. Percutaneous blood pumps can be advanced into a patient's vasculature along a guidewire. There is an ongoing need to provide alternative configurations of percutaneous blood pumps, and methods of loading the percutaneous blood pumps on a guidewire for advancement into a patient's vasculature.

### SUMMARY

This disclosure provides design, material, manufacturing method, and use alternatives for medical devices, such as a percutaneous blood pump.

One example is a percutaneous circulatory support device for use with a guidewire. The percutaneous circulatory support device includes a housing including an interior lumen, an exterior surface, a blood inlet, and a blood outlet that includes at least one blood outflow window. An impeller is disposed within the interior lumen. The impeller is configured to rotate relative to the housing to cause blood flow into the blood inlet, through the interior lumen of the housing, and out of the blood outlet. A motor is operatively coupled to the impeller. The motor is configured to rotatably drive the impeller. The exterior surface of the housing adjacent the at least one blood outflow window includes a marking.

Alternatively or additionally to any of the examples herein, in another example, the marking is an arrow.

Alternatively or additionally to any of the examples herein, in another example, the marking is laser printed onto the exterior surface of the housing.

Alternatively or additionally to any of the examples herein, in another example, the percutaneously circulatory support device includes a flexible cannula coupled to a distal end of the housing.

Alternatively or additionally to any of the examples herein, in another example, the coupling between the flexible cannula and housing forms a preformed curved portion, such that the percutaneous circulatory support device has an inner curvature and an outer curvature.

Alternatively or additionally to any of the examples herein, in another example, the at least one blood outflow window including a marking is disposed on the inner curvature of the percutaneous circulatory support device.

Alternatively or additionally to any of the examples herein, in another example, the percutaneous circulatory support device includes a channel extending from the interior lumen to the exterior surface of the housing.

Alternatively or additionally to any of the examples herein, in another example, the channel includes a ramped surface.

Alternatively or additionally to any of the examples herein, in another example, the channel includes a width greater than the guidewire.

Alternatively or additionally to any of the examples herein, in another example, the channel extends away from the at least one blood outflow window in a proximal direction.

Alternatively or additionally to any of the examples herein, in another example, the marking is disposed within the channel.

Alternatively or additionally to any of the examples herein, in another example, the plurality of blood outflow windows includes a second blood outflow window.

Alternatively or additionally to any of the examples herein, in another example, the channel extends proximally beyond the second blood outflow window.

Alternatively or additionally to any of the examples herein, in another example, the marking is disposed on an exterior surface of the housing adjacent the second blood outflow window.

Another example is a percutaneous circulatory support device for use with a guidewire. The percutaneous circulatory support device includes a housing having an interior lumen, an exterior surface, a blood inlet, and a blood outlet that includes at least one blood outflow window. An impeller is disposed within the interior lumen. The impeller is configured to rotate relative to the housing to cause blood flow into the blood inlet, through the interior lumen of the housing, and out of the blood outlet. A motor is operatively coupled to the impeller. The motor is configured to rotatably drive the impeller. The at least one blood outflow window includes a channel extending from the interior lumen to the exterior surface of the housing. The exterior surface adjacent the at least one blood outflow window includes a marking.

Alternatively or additionally to any of the examples herein, in another example, the marking is an arrow laser printed onto the exterior surface of the housing.

Alternatively or additionally to any of the examples herein, in another example, the channel includes a ramped surface.

Alternatively or additionally to any of the examples herein, in another example, the channel includes a width greater than the guidewire.

Alternatively or additionally to any of the examples herein, in another example, the channel extends away from the at least one blood outflow window in a proximal direction.

Yet another example is a method of loading a guidewire into a percutaneous circulatory support device. The method includes: inserting the guidewire into a distal end of a flexible catheter; advancing the guidewire through a housing, the housing comprising an interior lumen, an exterior surface, a blood inlet, and a blood outlet that includes at least one blood outflow window; advancing the guidewire to an impeller disposed within the interior lumen, the impeller configured to rotate relative to the housing to cause blood flow into the blood inlet, through the interior lumen of the housing, and out of the blood outlet; identifying a marking disposed on the exterior surface of the housing adjacent the at least one blood outflow window; and passing a proximal end of the guidewire out of one of the least one blood outflow window having the marking.

Alternatively or additionally to any of the examples herein, in another example, the marking is an arrow laser printed onto the exterior surface of the housing.

Alternatively or additionally to any of the examples herein, in another example, the blood outflow window includes a channel extending from the interior lumen to the exterior surface of the housing, the channel configured to receive and support the guidewire.

Alternatively or additionally to any of the examples herein, in another example, the channel includes a ramped surface.

Alternatively or additionally to any of the examples herein, in another example, the method includes manipulating the flexible catheter to direct the guidewire out of one of the at least one blood outflow window having the marking.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an exemplary catheter including a percutaneous blood pump;

FIG. 2 shows a distal end region of the catheter of FIG. 1 including the percutaneous blood pump;

FIG. 3 shows the distal end region of FIG. 2 including a guidewire;

FIG. 4 shows a perspective view of the distal end region of FIG. 2 including a blood outlet;

FIG. 5 is a perspective view of an example blood outlet; and

FIG. 6 is a perspective view of an example blood outlet.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

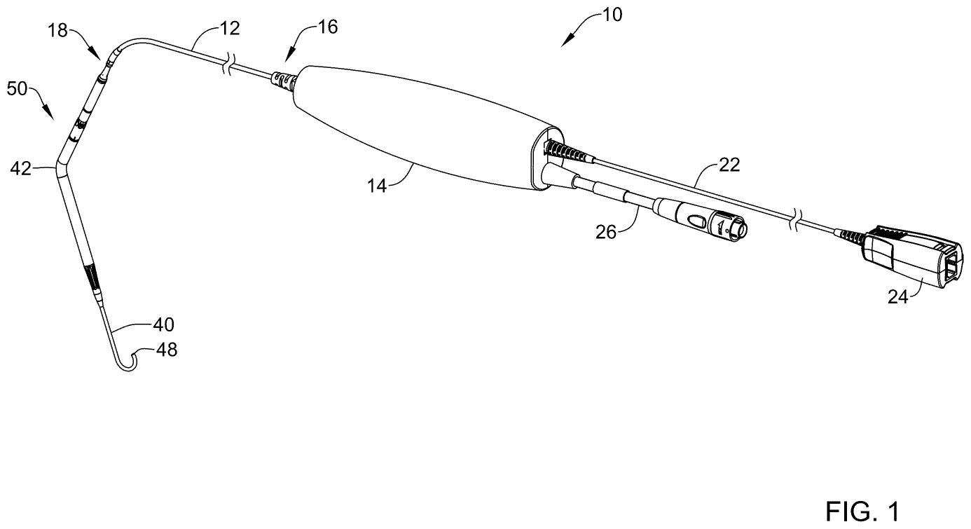

FIG. 1 illustrates a perspective view of a catheter 10 including a percutaneous blood pump 50 located at a distal end region thereof. The catheter 10 may be coupled to or include the blood pump 50, with an elongate shaft 12 of the catheter 10 extending proximally from the percutaneous blood pump 50 and a distal tip 40 extending distally from the blood pump 50. The distal tip 40 having and extending to a distal end 48. A distal portion of the blood pump 50 may be flexible, with a proximal portion being rigid. A bend 42 between the flexible and rigid portions of the blood pump 50 creates an inner curvature. A proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The catheter 10 may also include an extension 26 connectable to the controller for sending and/or receiving signals, such as from one or more sensors during operation of the blood pump 50.

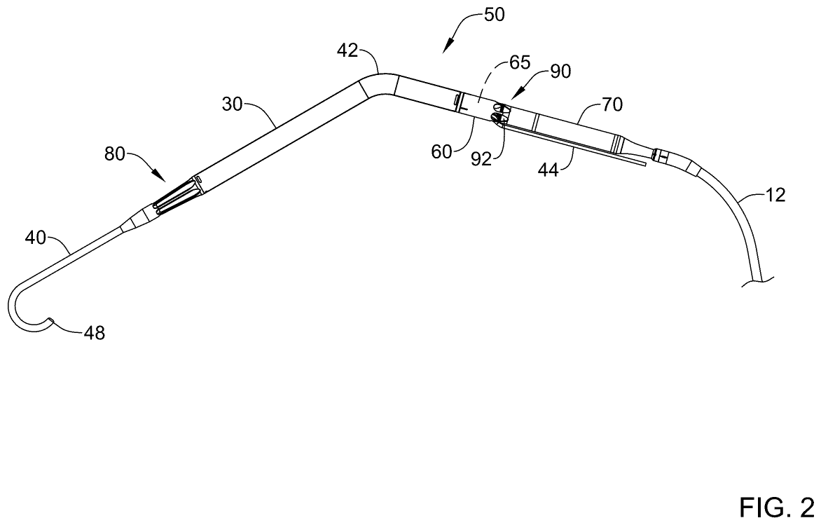

Additional features of the blood pump 50 are illustrated in FIG. 2. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows 92 arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

Additionally, shown in FIG. 2, the blood pump 50 may be supplied with or include a guidewire loading aid 44 to facilitate loading the catheter 10 onto a guidewire for introduction of the blood pump 50 into a vasculature. For example, the guidewire loading aid 44 may be a tubular member extending through at least a portion of the length of the blood pump 50 through which a guidewire may be routed when loading the catheter onto the guidewire. In some instances, the guidewire loading aid 44 may have a distal end (not shown) located within the blood pump 50 and a proximal end located exterior of the blood pump 50. A proximal end portion of the guidewire loading aid 44 may extend out through one of the outflow windows 92 of the blood outlet 90 and extend proximally therefrom. In some instances, the proximal end portion of the guidewire loading aid 44 may extend along an exterior of the motor housing 70, located proximal of the blood outlet 90. A guidewire, inserted through a guidewire lumen of the distal tip 40 (i.e., into the distal tip 40 through an opening at the distal end 48), may be guided into the lumen of the guidewire loading aid 44 and advanced proximally along the impeller assembly 65 and out through one of the outflow windows 92 of the blood outlet 90 within the lumen of the guidewire loading aid 44. Once the guidewire has been properly threaded through the blood pump 50, the guidewire loading aid 44 may be removed, leaving the guidewire tracked through the blood pump 50 such that the catheter 10 may be advanced over the guidewire into a vasculature.

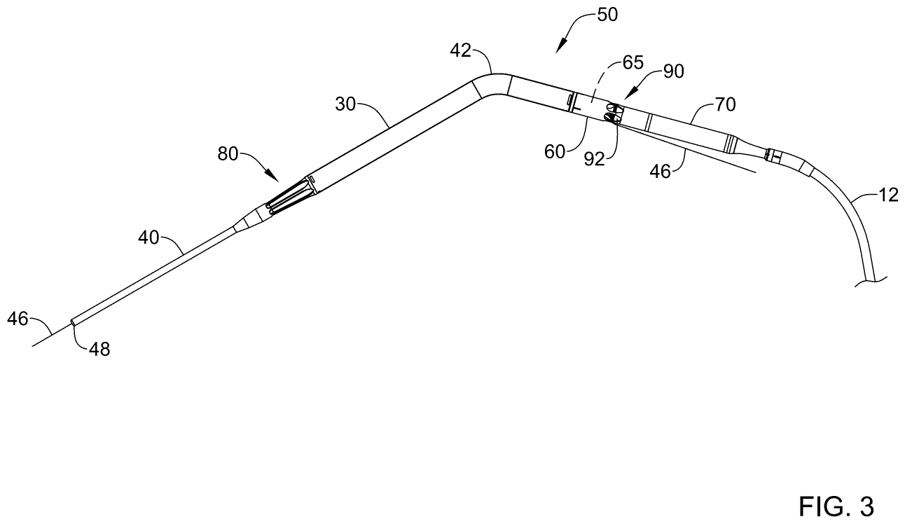

With reference to FIG. 3, in some instances it may be necessary to load a guidewire 46 into the blood pump 50 after removal of the loading aid 44, or in instances in which the blood pump 50 does not include a loading aid 44. For example, the guidewire 46 may need to be reloaded or repositioned after initial advancement of the catheter 10. Without the guidewire loading aid 44, a user still needs to advance the guidewire 46 through the correct or desired outflow window 92. In some embodiments, it may be desirable and/or necessary for the guidewire 46 to exit out of a particular outflow window 92 e.g. the outflow windows along the inner curvature (i.e., the radially inward concave side of the bend 42, or the side of the bend 42 having the smallest radius of curvature) of the blood pump 50. To aid in loading a guidewire 46 through the desired outflow windows 92, a user may manipulate the shape of the blood pump 50 at the bend 42, such that a guidewire extending through the blood pump 50 from the distal end 48 of the catheter tip 40, will be directed out of the desired outflow window 92. For example, the blood pump 50 if bent as shown in FIG. 3 will have a tendency to direct a guidewire out of the outflow windows 92 on the outer curvature (i.e., the radially outward convex side of the bend 42, or the side of the bend 42 having the greater radius of curvature) of the blood pump 50. The flexible portion 30 and bend 42 can be manipulated in any direction to aid in angling the guidewire for loading, and then returned to initial positioning for advancement of the catheter 10 into a patient's vasculature.

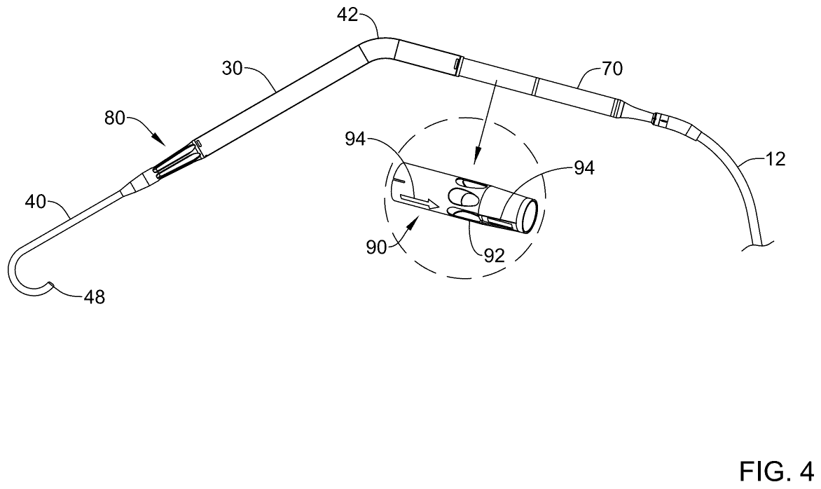

As shown in FIG. 4, the blood outlet 90, defined by the impeller housing 60, includes a marking 94 at (e.g., axially aligned with) one of the plurality of outflow windows 92. For instance, one of the plurality of windows 92 may include a marking 94 axially aligned with the window 92, while the reminder of the windows 92 extending around the circumference of the blood pump 50 may be devoid of a marking 94. The blood outlet 90 of the impeller housing 60 is shown in an enlarged view, noting that the impeller housing 60 has been rotated slightly from the cannula to better illustrate the marking 94. The marking 94 may be located distal of and axially aligned with the outflow window 92, be located proximal of and axially aligned with the outflow window 92, extend around the periphery of the outflow window 92 or a portion thereof, or otherwise be indicative of the designated outflow window 92. The marking 94 may indicate which one of the plurality of outflow windows 92 a user can advance a guidewire out of, or which one of the plurality of outflow windows 92 the guidewire is intended to be advanced out of. There may be any number of markings 94 on a given one of the plurality of windows 92, as desired. For example, the outflow window 92 may include a first marking 94 located distal of and axially aligned with the outflow window 92 and a second marking 94 located proximal of and axially aligned with the outflow window 92. Furthermore, it is understood that more than one of the plurality of windows 92 (but less than all of the plurality of windows 92) may include a marking 94 depending on the individual configuration of the blood pump 50. For instance, in some instances two adjacent windows 92 may include a marking 94, while the remainder of the windows 92 extending around the circumference of the blood pump 50 may be devoid of a marking 94. The markings 94 may be laser printed, etched, pad printed or the like onto the outer surface of the housing of the blood outlet 90.

The designated outflow window 92 having the associated marking 94 may be arranged on the inner curvature (i.e., the radially inward concave side of the bend 42, or the side of the bend 42 having the smallest radius of curvature) of the blood pump 50. In other words, the designated outflow window 92 having the associated marking 94 may be circumferentially oriented to be on the same side of the blood pump 50 as the inner curvature (i.e., the radially inward concave side of the bend 42, or the side of the bend 42 having the smallest radius of curvature) of the blood pump 50.

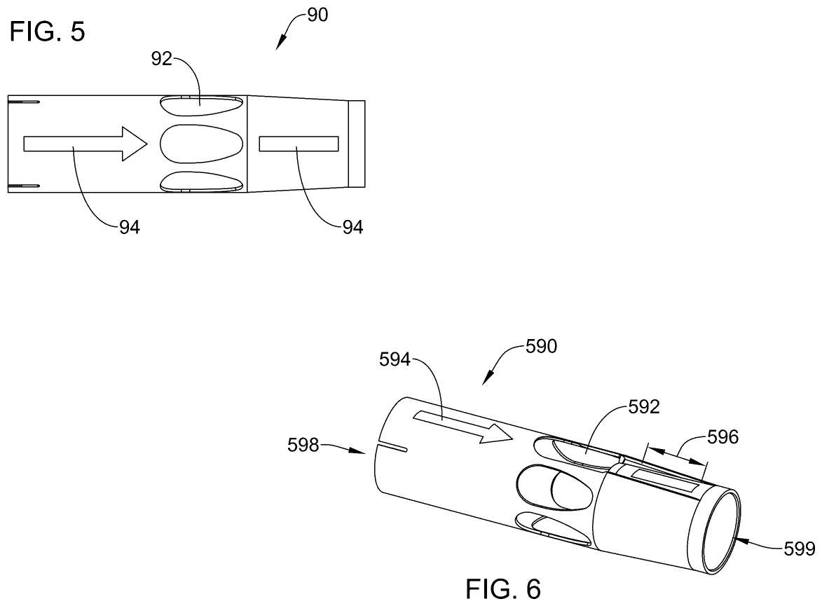

FIG. 5 illustrates a blood outlet 90 as in FIG. 4, wherein the blood outlet 90 has one marking 94 on (e.g., aligned with) one of the outflow windows 92 while the remainder of the windows 92 extending around the circumference of the blood outlet 90 may be devoid of a marking. The marking 94 indicates to a user which window 92 to advance a guidewire out of, and has an arrow pointing in the direction that the guidewire should be advanced out of the window 92. In some embodiments, there may be multiple markings 94 at multiple outflow windows 92. For example, in some embodiments, any of the outflow windows 92 on the inner curvature of the blood pump 50 may be designated for the guidewire to be advanced out of.

FIG. 6 shows another variation of a blood outlet 590 of the blood pump 50. With reference to FIG. 6, in some embodiments, the blood outlet 590 may include a recess or channel 596 extending proximally from one of the outlet windows 592 toward the proximal end 599 of the blood outlet 590 configured to receive and support a guidewire extending proximally from the outlet window 592. The channel 596 may have a width greater than the guidewire (i.e., greater than the diameter of the guidewire). The channel 596 may include a ramped surface configured to support the guidewire as is exits the outflow window 592 in a proximal direction. Referring back to FIG. 3, when the guidewire 46 extends out of the blood outlet window 592, it may be subjected to high contact forces at the outflow window 592 of the blood outlet 590. Thus, it may be advantageous to provide a channel which reduces the risk of damaging the guidewire. In some embodiments, the guidewire 46 may have a coating covering at least a portion of the guidewire 46. The channel 596 may be configured to prevent the guidewire 46 with or without a coating from being abraded or damaged by frictional contact with the housing 590 (e.g., the peripheral edge of the outflow window 592) as the guidewire exits an outflow window 592, thus reducing the potential for damage to the guidewire. In some embodiments, the channel 596 and/or outflow window 592 may include a rounded edge to prevent the guidewire 46 from being damaged as it exits the blood outlet window 592.

As shown in FIG. 6, the channel 596 extends from a blood outflow window 592 toward the proximal end 599 of the blood outlet 590. In other embodiments, the channel 596 may be located on other parts of the blood outlet 590. It should be understood that there may be any number of channels 596 disposed at any location on the blood outlet 590. In some embodiments, there may be multiple channels 596 at multiple blood outlet windows 592.

With continued reference to FIG. 6, the blood outlet 590 may include marking 594. A marking 594 may extend between the distal end 598 of the blood outlet and one of the blood outflow windows 592 and/or a marking 594 may extend between one of the blood outflow windows 592 and the proximal end 599 of the blood outlet 590. The marking 594 may indicate to a user which outflow window 592 to advance a guidewire out of. The marking 594 may be laser printed, etched, pad printed or the like onto the outer surface of the blood outlet 590. The marking 594 can be disposed within or adjacent the channel 596. It should be understood that there may be any number and configuration of channels 596 and markings 594 disposed around the circumference of the blood outlet 590. In some instances, the marking 594 may be an arrow pointing in the direction that the guidewire should extend from the designated blood outflow window 592.

It will be understood that any relative dimensions described in association with the above figures are illustrative only, and that other dimensions of various components of the medical device are contemplated. The materials that can be used for the various components of the percutaneous circulatory support device (and/or other systems or components disclosed herein) and the various elements thereof disclosed herein may include those commonly associated with medical devices. For simplicity purposes, the following discussion refers to the percutaneous circulatory support device (and variations, systems or components disclosed herein). However, this is not intended to limit the devices and methods described herein, as the discussion may be applied to other elements, members, components, or devices disclosed herein.

In some embodiments, the percutaneous circulatory support device (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the percutaneous circulatory support device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the percutaneous circulatory support device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the percutaneous circulatory support device (and variations, systems or components thereof disclosed herein) to achieve the same result.

In some embodiments, the percutaneous circulatory support device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, components of the medical device can be blended with a liquid crystal polymer (LCP). For example, the mixture can contain up to about 6 percent LCP.

In some embodiments, the percutaneous circulatory support device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A percutaneous circulatory support device for use with a guidewire, comprising:
a housing comprising an interior lumen, an exterior surface, a blood inlet, and a blood outlet that includes at least one blood outflow window;
an impeller disposed within the interior lumen, the impeller configured to rotate relative to the housing to cause blood flow into the blood inlet, through the interior lumen of the housing, and out of the blood outlet;
a motor operatively coupled to the impeller, the motor configured to rotatably drive the impeller,
wherein the exterior surface of the housing adjacent the at least one blood outflow window comprises a marking.

2. The percutaneous circulatory support device of claim 1, wherein the marking is an arrow.

3. The percutaneous circulatory support device of claim 1, wherein the marking is laser printed onto the exterior surface of the housing.

4. The percutaneous circulatory support device of claim 1, further comprising a flexible cannula coupled to a distal end of the housing.

5. The percutaneous circulatory support device of claim 4, wherein the coupling between the flexible cannula and housing forms a preformed curved portion, such that the percutaneous circulatory support device has an inner curvature and an outer curvature.

6. The percutaneous circulatory support device of claim 5, wherein the at least one blood outflow window comprising a marking is disposed on the inner curvature of the percutaneous circulatory support device.

7. The percutaneous circulatory support device of claim 1, further comprising a channel extending from the interior lumen to the exterior surface of the housing.

8. The percutaneous circulatory support device of claim 7, wherein the channel includes a ramped surface.

9. The percutaneous circulatory support device of claim 7, wherein the channel includes a width greater than the guidewire.

10. The percutaneous circulatory support device of claim 7, wherein the channel extends away from the at least one blood outflow window in a proximal direction.

11. A percutaneous circulatory support device for use with a guidewire, comprising:
a housing comprising an interior lumen, an exterior surface, a blood inlet, and a blood outlet that includes at least one blood outflow window;
an impeller disposed within the interior lumen, the impeller configured to rotate relative to the housing to cause blood flow into the blood inlet, through the interior lumen of the housing, and out of the blood outlet;
a motor operatively coupled to the impeller, the motor configured to rotatably drive the impeller,
wherein the at least one blood outflow window includes a channel extending from the interior lumen to the exterior surface of the housing, the exterior surface adjacent the at least one blood outflow window comprising a marking.

12. The percutaneous circulatory support device of claim 11, wherein the marking is an arrow laser printed onto the exterior surface of the housing.

13. The percutaneous circulatory support device of claim 11, wherein the channel includes a ramped surface.

14. The percutaneous circulatory support device of claim 11, wherein the channel includes a width greater than the guidewire.

15. The percutaneous circulatory support device of claim 11, wherein the channel extends away from the at least one blood outflow window in a proximal direction.

16. A method of loading a guidewire into a percutaneous circulatory support device, the method comprising:
inserting the guidewire into a distal end of a flexible catheter;
advancing the guidewire through a housing, the housing comprising an interior lumen, an exterior surface, a blood inlet, and a blood outlet that includes at least one blood outflow window;
advancing the guidewire to an impeller disposed within the interior lumen, the impeller configured to rotate relative to the housing to cause blood flow into the blood inlet, through the interior lumen of the housing, and out of the blood outlet;
identifying a marking disposed on the exterior surface of the housing adjacent the at least one blood outflow window; and
passing a proximal end of the guidewire out of one of the least one blood outflow window having the marking.

17. The method of claim 16, wherein the marking is an arrow laser printed onto the exterior surface of the housing.

18. The method of claim 16, wherein the blood outflow window includes a channel extending from the interior lumen to the exterior surface of the housing, the channel configured to receive and support the guidewire.

19. The method of claim 18, wherein the channel includes a ramped surface.

20. The method of claim 16, further comprising, manipulating the flexible catheter to direct the guidewire out of one of the at least one blood outflow window having the marking.
